(19) United States
(12) Patent Application Publication
(10) Pub. No.: US 2007/0224604 A1
(43) Pub. Date: Sep. 27, 2007

(54) Method of Determining the Presence and/or Concentration of Substances of Interest in Fluids

(76) Inventors: Patricia Connolly, Glasgow (GB); John Fuller, Perthshire (GB)

Correspondence Address: KAPLAN GILMAN GIBSON & DERNIER L.L.P., 900 ROUTE 9 NORTH, WOODBRIDGE, NJ, 07095, US

(21) Appl. No.: 11/570,844

(22) PCT Filed: Jun. 20, 2005
(86) PCT No.: PCT/GB05/02427

§ 371 Date: Apr. 19, 2007

(30) Foreign Application Priority Data
Jun. 19, 2004 (GB) 0413752.7

(51) Int. Cl. C12Q 1/68; G01N 33/551

(52) U.S. Cl. 435/6; 436/524

(57) ABSTRACT

Magnetic particles coated with specific antibodies for substances of interest are introduced into a fluid sample. Over time and with agitation, the magnetic particles become attached to the substance of interest within the fluid. The fluid is then introduced to a magnetic field gradient provided between two magnets (1, 5). the resultant change to the magnetic filed between the magnets (1, 5) is determine by a Hall Effect device (3) or a plurality of Hall Effect devices (3). This enables the presence and/or concentration of the substance of interest in the sample to be determined.

The present invention relates to a method of determining the presence and/or concentration of substances of interest in fluids, and particularly although not exclusively the presence and/or concentration of substances of interest in biological fluids including measurement in a living body, such as a human body.

In many medical, biological, manufacturing and other systems there is a requirement to determine the presence and/or concentration of various substances in fluids, including, but not limited to molecules such as proteins, hormones or DNA. In the normal course of analysis immunoassay systems are employed to measure such molecules. These immunoassays rely on the presence of a tagged antibody or probe (ligand) which adheres to or binds to a molecule of interest. The presence of the tagged probe is detected and the quantity of probe detected related to the concentration of the molecule under analysis. Multiple probe systems with a capture probe or antibody and a second tagged probe or antibody to reveal the captured molecule are common. Probes have been used with tags that are radioactive, enzymatic, fluorescent, chemiluminescent and spectrophotmetric or colourimetric. End points of tagged probe measurement can therefore be revealed in a variety of systems include spectrophotometric, electrochemical, radioactive, colourimetric, amperometric or potentiometric.

Magnetic beads have been employed in multiple probe systems as a solid phase for the capture probe, providing a highly mobile bead system with high surface area for capture probe attachment [1]. Secondary probes or antibodies can then be added after molecular attachment to the capture probe and in the commonest application a magnetic field is then used to draw together the beads allowing a concentrate to form where the level of the tag can be measured. This can be read directly, if, for example, the tag is fluorescent or can be read by an indirect method whereby a solution is introduced to react with the probe tag producing a measurable effect (such as the production of light by chemiluminescent reactions with the tag).

A number of documents, U.S. Pat. No. 6,046,585, U.S. Pat. No. 6,275,031, U.S. Pat. No. 6,437,563 & U.S. Pat. No. 6,483,303, (all assigned to Quantum Dynamics Inc) all disclose using an immunoassay to determine the presence of magnetic particles complexed with substances of interest in a sample. A magnetic field is applied to the sample and the magnetic particles are thereby caused to oscillate at the excitation frequency in the manner of a dipole to create their own fields. The fields are inductively coupled to at least one sensor to produce a useful indication of the presence of said magnetic particles.

It is an object of the present invention to provide an alternative method of determining the presence and/or concentration of substances of interest in a fluid. It is an object of embodiments of the present invention to enable determination of the presence and/or concentration of substances in low volumes of fluid, typically less than 5 μL. It is a preferred outcome of the invention that such assays should take place under rapid capillary and/or turbulent flow conditions to reduce assay times.

According to an aspect of the present invention there is provided a method of determining the presence and/or concentration of one or more substances of interest in a fluid, the method comprising the steps of: attaching a magnetic particle to the or each substance of interest in the fluid and introducing the fluid into an inhomogeneous magnetic field having a field gradient and thereby determining the presence and/or concentration of magnetic particles in the fluid thereby to determine the presence and/or concentration of the one or more substances of interest.

Hitherto determination of the presence and/or concentration of magnetic particles in this manner has not been employed to infer the presence of substances of interest attached to those particles. Employing this technique enables rapid analysis of a fluid, and effective analysis of very small volumes of fluid.

The fluid may be a liquid or gas, and may be a biological fluid such as a body fluid.

Substances of interest may include naturally occurring substances, substances that are the result of a chemical or biological reaction, such as drug by-products, and substances introduced into a fluid sample. The substance may be a compound, especially a molecule and could be, for example a protein, hormone or DNA section.

By “magnetic” particles is to be understood particles of non-zero magnetic susceptibility. The or each magnetic particle may be ferromagnetic, diamagnetic, paramagnetic or superparamagnetic. A homogeneous or heterogeneous mixture of such particles may be employed. In one embodiment the or each particle is formed from iron oxide. Particles of size in the range 5 nanometers to 100 micrometers may be used or in some embodiments particles of size in the range 5 nanometers to 50 micrometers may be used.

The or each particle may be attached to a substance of interest by means of a further substance, which shall be referred to as a bonding substance. The or each particle may be coated with the bonding substance. The bonding substance may be a protein, and in some embodiments it is an antibody or probe (ligand).

The or each magnetic particle may be coated with a material to facilitate adherence of a bonding substance to the particle. A suitable coating material is polystyrene.

Magnetic particles may be attached to substances of interest prior to their introduction into a fluid, for example in the case of a drug injected into a human body. Alternatively magnetic particles coated with an appropriate bonding substance may be introduced into a fluid containing a substance of interest so that they will become attached to the substance of interest, for example appropriately coated magnetic particles could be injected into a human body in order to identify the presence and/or concentration of specific drug by-products.

By appropriate selection of the bonding substance it is possible to arrange for magnetic particles to attach to a variety of substances of interest. The or each magnetic particle may be arranged so that it can only become attached to a single unit of a substance of interest, for example a single molecule. As such each particle may be provided with a single antibody or capture probe.

It is possible to distinguish between magnetic particles having different characteristics in a fluid. By using magnetic particles having common known characteristics attached to a first bonding substance and further magnetic particles having different known characteristics attached to other bonding substances it is possible to determine the presence and/or concentration of more than one substance of interest in a given fluid. Any suitable characteristic of the magnetic particles may be altered, including size, shape and magnetic susceptibility.

In some instances it is also possible to distinguish between magnetic particles with the same or similar characteristics attached to different substances of interest in a fluid, by virtue of differing characteristics of the substance of interest such as a mass.

In one embodiment the effect of the fluid to be analysed on the magnetic field is measured using a magnetic field sensor to determine magnetic particles in the fluid, and thereby the presence and/or concentration of substances of interest. Preferably the magnetic field gradient is along a line in space. When magnetic particles in a fluid are introduced into the field gradient they will experience a force. The force experienced by each particle will depend upon its characteristics. Where a fluid containing a number of magnetic particles which respond differently to a magnetic field is placed into an inhomogeneous magnetic field the different particles are subject to different forces and will thus tend to migrate to different regions of the field. The magnetic particles present in the fluid will also influence the magnetic field in different ways. With knowledge of the way in which different particles introduced into a fluid influence magnetic field it is possible to infer their presence and also the concentration of the particles in the fluid. In one embodiment particles of differing susceptibility are present in a fluid and the fluid is introduced into a magnetic field gradient. Particles of the same susceptibility will tend to migrate to the same position within the field gradient. The amount of particles present at any one point will affect the field strength at that point. By measuring the strength of magnetic field in the region of the fluid along the field gradient it is possible to determine the presence and quantity of magnetic particles of differing susceptibility and thereby the concentration of particles of the same or similar susceptibility in the fluid sample.

Field gradients in the range 50 to 200 Tesla per metre may be typically employed and not excluding other field gradients. Permanent magnets having shaped pole pieces may typically be used to provide a magnetic field gradient.

In yet another embodiment the particles can be detected in a classic ‘displacement assay’ or ‘flow displacement assay’. Here the particles are immobilised on a surface but are able to bond to a substance of interest though a specific bonding substance such as an antibody. A sample is introduced to this surface which contains the substance of interest in an unknown quantity. Competition for the binding site on the particle from the substance of interest in the sample will release the magnetic particles into solution in proportion to the concentration of the substance of interest in the sample. In the present invention, the released particles experience a magnetic field gradient and hence are concentrated at a point determined by their susceptibility. This concentration varies the magnetic field and creates a point of high field density. A magnetic sensor is placed at the point of highest field density to detect the particles thus greatly increasing the sensitivity of measurement. The sensor may be a Hall Sensor, or any other sensitive magnetic measurement sensor. The immobilised particles can be bound via any suitable bonding substance to substances of interest, multiple layers of different bonding substances can be used to create suitable sites for competition from substances of interest in the sample.

Advantageously there is no requirement for a secondary antibody capture site to “collect” the particles together for sensing. This makes the disposable element simpler and cheaper. Additionally, there is no requirement for complex alignment systems between sensor and disposable to give accuracy and consistency. The magnetic field gradient automatically concentrates all freed particles in the sensing area. Furthermore, the use of NdFeb (Neodymium Iron Boron) permanent magnets together with correctly designed pole pieces allows very high field densities to be created. This gives much greater sensitivity with no power input which is essential for small, low cost point of care systems

In the prior art, a displacement assay is used in the analytical system but measurement is made by a complex oscillating coil system, and an antibody capture site for particles. This cannot create the same level of field density, even with very high power inputs, and hence sensitivity is compromised. This results in more complex manufacture for both the sensing system and the disposable test element. Therefore it is not suitable for small, point of care machines.

Furthermore prior art methods using magnetic particles utilise an immunoassay and adjacent magnets to move and measure spatial separation of magnetic particles the present invention utilises the properties of a magnetic field gradient, knowledge of the effect of the particles on total field and a sensitive magnetic sensor to be able to determine the particle flow, both spatially and temporally, during the assay and to thereby determine the quantity of the substance of interest present in the sample being examined.

It should be understood that the ability to understand and utilise magnetic field gradients with magnetic particles in the present invention may also be employed to maximise the immunoassay efficiency and performance. In this manner the present invention may utilise oscillating and other magnetic field arrangements to enhance particle-sample interactions and/or to manipulate flow rates during assays through manipulation of the particles. For example, a magnetic field may be introduced that will directly cause the magnetic particles to coagulate or cluster within a device slowing flow rates in the sample. Higher amplification may be used to achieve greater sensitivity. Furthermore, the method may include the application of an oscillating magnetic field to a solution of the fluid to be analysed and the magnetic particles to ensure mixing before introducing the fluid into the said inhomogeneous field with a field gradient.

According to another aspect of the present invention there is provided a method of determining the presence and/or concentration of one or more substances of interest in a fluid, the method comprising the steps of: introducing a fluid to be analysed is into a chamber containing magnetic particles bound with probes specific for the capture of the molecule of interest; mixing the magnetic particles with the fluid by the application of an oscillating magnetic field to the chamber to thereby determine the presence and/or concentration of magnetic particles in the fluid thereby to determine the presence and/or concentration of the one or more substances of interest.

The fluid may be a liquid or gas, and may be a biological fluid such as a body fluid.

Substances of interest may include naturally occurring substances, substances that are the result of a chemical or biological reaction, such as drug by-products, and substances introduced into a fluid sample. The substance may be a compound, especially a molecule and could be, for example a protein, hormone or DNA section.

By “magnetic” particles is to be understood particles of non-zero magnetic susceptibility. The or each magnetic particle may be ferromagnetic, diamagnetic, paramagnetic or superparamagnetic. A homogeneous or heterogeneous mixture of such particles may be employed. In one embodiment the or each particle is formed from iron oxide. Particles of size in the range 5 nanometers to 100 micrometers may be used or in some embodiments particles of size in the range 5 nanometers to 50 micrometers may be used.

The or each particle may be attached to a substance of interest by means of a further substance, which shall be referred to as a bonding substance. The or each particle may be coated with the bonding substance. The bonding substance may be a protein, and in some embodiments it is an antibody or probe (ligand).

The or each magnetic particle may be coated with a material to facilitate adherence of a bonding substance to the particle. A suitable coating material is polystyrene.

Magnetic particles may be attached to substances of interest prior to their introduction into a fluid, for example in the case of a drug injected into a human body. Alternatively magnetic particles coated with an appropriate bonding substance may be introduced into a fluid containing a substance of interest so that they will become attached to the substance of interest, for example appropriately coated magnetic particles could be injected into a human body in order to identify the presence and/or concentration of specific drug by-products.

By appropriate selection of the bonding substance it is possible to arrange for magnetic particles to attach to a variety of substances of interest. The or each magnetic particle may be arranged so that it can only become attached to a single unit of a substance of interest, for example a single molecule. As such each particle may be provided with a single antibody or capture probe.

It is possible to distinguish between magnetic particles having different characteristics in a fluid. By using magnetic particles having common known characteristics attached to a first bonding substance and further magnetic particles having different known characteristics attached to other bonding substances it is possible to determine the presence and/or concentration of more than one substance of interest in a given fluid. Any suitable characteristic of the magnetic particles may be altered, including size, shape and magnetic susceptibility.

In some instances it is also possible to distinguish between magnetic particles with the same or similar characteristics attached to different substances of interest in a fluid, by virtue of differing characteristics of the substance of interest such as a mass.

The chamber may have a volume of less than 10 μL, preferably less than 5 μL. The magnetic particles are mixed with the fluid by the application of an oscillating magnetic field to the chamber. A sensitive detector of magnetic field, such as a Hall Effect probe, is used to detect movement of magnetic particles throughout the fluid. As the probes and particles bind to the substances of interest the mass of particles will start to move together in the oscillating field creating a magnetic field pattern which will be distributed in a unique mode throughout the chamber. This can be detected by the magnetic field sensor and both its distribution and time-spatial development can be used to determine the concentration of the substance of interest in the fluid.

In a variation to this embodiment capture antibodies, specific to the analyte of interest, are spatially immobilised at one part of the chamber and the magnetic particles are coated with a second antibody (tag probe) specific to the substance of interest. As the molecule binds to the capture and tag antibodies, the magnetic field due to the presence of bound particles increases at the location in the chamber of the capture antibodies and can be specifically measured at this site, thereby to infer the concentration of the substance of interest. In a further variation an applied magnetic field is used to specifically concentrate particles of different magnetic susceptibilities in the sample. This allows for the measurement of multiple substances, molecules or analytes in a sample, such as a blood sample, where each different type of magnetic particle carries a different capture probe.

Additional sensitivity may be gained by providing two chambers and sensing areas, one of which contains the fluid and particles, the other being a control chamber. By sensing the two chambers together and by taking a differential signal the system becomes immune to external magnetic influence as signals that affect both sensors together are effectively cancelled out.

In order that the invention may be more clearly understood embodiments thereof will now be described by way of example with reference to the accompanying drawings of which:

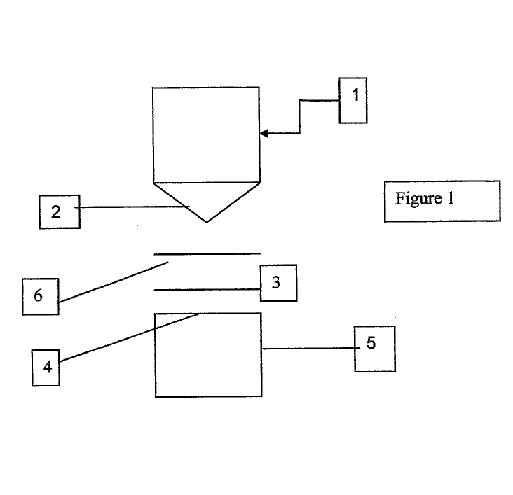

FIG. 1 is a schematic view of apparatus for performing an embodiment of the invention.

In one embodiment of the present invention two types of iron oxide particles are provided, a first type of a first size and susceptibility and a second type of a second size and susceptibility. Both types of particles are coated with polystyrene to provide an inert surface to the particle. Subsequently the first type of particles are coated with a first antibody arranged to bond to a first substance of interest, and the second type of particles are coated with a second antibody arranged to bond to a second substance of interest.

Both types of particles are then introduced into a fluid in which it is desired to detect the presence and/or quantity of the first and second substances of interest. Over time and with agitation of the fluid the first type of particles will become attached to the first substance of interest and the second type of particles will be become attached to the second substance of interest. Sufficient quantities of each type of particle are introduced to ensure that a particle becomes bonded to each substance of interest.

The fluid is subsequently analysed using the apparatus illustrated in FIG. 1. The apparatus comprises two spaced apart rare earth permanent magnets 1, 5 having substantially parallel opposed flat facing surfaces 7 on one of which is mounted a shaped soft ion pole piece 2. The pole piece is substantially triangular in cross-section presenting a wedge shaped profile extending away from its associated magnet 1, directed towards the other magnet 5. The magnets 1, 5 and pole piece 2 are operative to generate a magnetic field gradient in the space between the two magnets. The field gradient extends in the direction indicated as 3 in FIG. 1.

The magnet system further includes a linear array of Hall Effect devices 6 extending between the magnets in direction 3, or alternatively may include a single Hall device moveable between the magnets along direction 3. in either case the Hall Effect devices or Hall Effect device operates to measure magnetic field strength between the magnets 1, 5 along direction 3.

The fluid to be analysed is introduced in a container, or in a living body, into a region of magnetic field gradient between two magnets 1, 5 and the resultant change in magnetic field, due to any of the above assays utilising magnetic particles, between the magnets along the direction 3 is measured by the Hall Effect device 3, or devices. Pole pieces 2, 4 shape and control the magnetic field.

Apparatus suitable for analysing a fluid sample containing substances tagged with magnetic particles is also disclosed in WO 02/088696.

If as described above, two types of particle, attached to respective substances of interest are utilised in this device, they will tend to migrate to two discrete points along the axis 3 where their presence will influence the measured magnetic field at that point. These points and the effect of the presence of particles on the magnetic field can be determined empirically. By calibration of the apparatus it is possible to determine the presence and/or concentration of the substances associated with each type of particle in the fluid sample. Of course any number of different types of particle may be employed in a single fluid to identify a corresponding number of substances of interest.

In a variation of this embodiment, the particles can be utilised in a classic ‘displacement assay’ or ‘flow displacement assay’. In such embodiments, the particles are immobilised on a surface. As previously, the particles are able to bond to the substance of interest via their attachment to specific antibodies. When a sample fluid is introduced on to the surface, competition for the bonding site on the particle from the substance of interest results in the release of the particles into solution in an amount in proportion to the concentration of the substance of interest in the sample fluid. The released particles are exposed to a field gradient as described above and accordingly, the particles become concentrated at a particular point along the field gradient according to their magnetic susceptibility. At this point, the field density is therefore increased. The change in the field may be measured using a Hall Effect device or any other suitable magnetic sensor. As above, suitable calibration enables the determination of the presence and/or concentration of the substance(s) of interest in the sample.

In an alternative embodiment, one or more types of iron oxide particles of the type described above are provided in a chamber. The chamber would typically have a volume of less than 10 μL or in some embodiments 5 μL. A fluid containing one or more substances of interest is then introduced to the chamber. After the introduction of the fluid to the chamber, an oscillating magnetic field is applied to the chamber which has the effect of mixing the particles with the fluid. The particle fluid mix can then be analysed as described in the first embodiment above to determine the presence and or concentration of the one or more substances of interest.

In a further alternative embodiment, one or more types of iron oxide particles of the type described above are provided in a chamber. The chamber would typically have a volume of less than 10 μL or in some embodiments 5 μL. A fluid containing one or more substances of interest is then introduced to the chamber. After the introduction of the fluid to the chamber, an oscillating magnetic field is applied to the chamber which has the effect of mixing the particles with the fluid. Continued application of the oscillating magnetic field causes the mass of particles to move together in the oscillating field creating a magnetic filed pattern distributed in a unique mode throughout the chamber. The magnetic filed pattern is detected by a suitable magnetic sensor, such as a Hall Effect device. With suitable calibration, the distribution and the time-spatial development of the pattern may be use to determine the presence and/or concentration of substances of interest in the fluid.

In a variation of the above embodiments, capture antibodies are immobilised at a specific location within the chamber, the capture antibodies specific to the substance of interest. The magnetic particles are coated with a second antibody (tag probe) specific to the substance of interest. The substance of interest binds to both the capture antibodies and the tag probe and accordingly, the magnetic particles become immobilised at the location of the capture antibodies. A suitable magnetic field sensor, such as a Hall Effect device, is then used to measure the magnetic field at this location. The field strength can, through suitable calibration, be used to determine the presence and/or concentration of the substance of interest in a fluid. Alternatively, rather than immobilised capture antibodies, an applied magnetic field can be use to specifically concentrate particles of particular susceptibilities at particular locations within the chamber. This allows for the determination of the presence and/or concentration of a plurality of different substances in a sample, provided each different type of magnetic particle carries a different capture probe.

In a further variation of the above embodiments, to gain additional sensitivity, two similar chambers may be used, one containing the fluid and the particles, the other being a control chamber. By sensing the two chambers together and taking a differential signal, the system is not affected by external magnetic fields as these effect both chambers equally and are thus cancelled out.

The above embodiments are described by way of example only, many variations are possible without departing from the present invention.

### REFERENCES

1. Application of magnetic techniques in the field of drug discovery and biomedicine Z M Saiye, S D Telang and C N Ramchand, BioMagnetic Research and Technology Volume 1

1. A method of determining the presence and/or concentration of one or more substances of interest in a fluid, the method comprising the steps of: attaching a magnetic particle to the or each substance of interest in the fluid and introducing the fluid into an inhomogeneous magnetic field having a field gradient and thereby determining the presence and/or concentration of magnetic particles in the fluid thereby to determine the presence and/or concentration of the one or more substances of interest.

2. A method as claimed in claim 1 wherein the fluid is a liquid.

3. A method as claimed in claim 1 wherein the fluid is a biological fluid such as a body fluid.

4. A method as claimed in claim 1 wherein the fluid is a gas.

5. A method as claimed in claim 1 wherein the substance is a compound.

6. A method as claimed in claim 1 wherein the substance is any one of a protein, a hormone or a DNA section.

7. A method as claimed in claim 1 wherein magnetic particles having common known characteristics attached to a first bonding substance and further magnetic particles having different known characteristics attached to other bonding substances are used to determine the presence and/or concentration of more than one substance of interest in a given fluid.

8. A method as claimed in claim 1 wherein differing characteristics of the substance of interest are used to distinguish between magnetic particles with the same or similar characteristics attached to different substances of interest.

9. A method as claimed in claim 1 wherein the effect of the fluid to be analysed on the magnetic field is measured using a magnetic field sensor to determine magnetic particles in the fluid, and thereby the presence and/or concentration of substances of interest.

10. A method as claimed in claim 1 wherein the particles are detected in a classic ‘displacement assay’ or ‘flow displacement assay’.

11. A method as claimed in claim 10 wherein the released particles enter a magnetic field gradient and are concentrated at a particular point in the gradient determined by their susceptibility.

12. A method as claimed in claim 11 wherein a magnetic sensor is placed at the point of highest field density.

13. A method as claimed in claim 1 wherein the sensor is a Hall Sensor.

14. A method as claimed in claim 10 wherein an oscillating magnetic field arrangement is applied to enhance particle-sample interactions and/or to manipulate flow rates.

15. A method as claimed in claim 10 wherein a field is introduced that will directly cause the magnetic particles to coagulate or cluster within a device, slowing flow rates in the sample.

16. A method as claimed in claim 1 wherein an oscillating magnetic field is applied to a solution of the fluid to be analysed and the magnetic particles to ensure mixing before introducing the fluid into the said inhomogeneous field with a field gradient.

17. A method of determining the presence and/or concentration of one or more substances of interest in a fluid, the method comprising the steps of: introducing a fluid to be analysed into a chamber containing magnetic particles bound with probes specific for the capture of the molecule of interest; mixing the magnetic particles with the fluid by the application of an oscillating magnetic field to the chamber to thereby determine the presence and/or concentration of magnetic particles in the fluid thereby to determine the presence and/or concentration of the one or more substances of interest.

18. A method as claimed in claim 17 wherein the fluid is a liquid.

19. A method as claimed in claim 17 wherein the fluid is a biological fluid such as a body fluid.

20. A method as claimed in claim 17 wherein the fluid is a gas.

21. A method as claimed in claim 17 wherein the substance is a compound.

22. A method as claimed in claim 17 wherein the substance is any one of a protein, a hormone or a DNA section.

23. A method as claimed in claim 17 wherein magnetic particles having common known characteristics attached to a first bonding substance and further magnetic particles having different known characteristics attached to other bonding substances are used to determine the presence and/or concentration of more than one substance of interest in a given fluid.

24. A method as claimed in claims 17 wherein differing characteristics of the substance of interest are used to distinguish between magnetic particles with the same or similar characteristics attached to different substances of interest.

25. A method as claimed in claim 17 wherein the effect of the fluid to be analysed on the magnetic field is measured using a magnetic field sensor to determine magnetic particles in the fluid, and thereby the presence and/or concentration of substances of interest.
